(19) United States
(12) Patent Application Publication
(10) Pub. No.: US 2023/0355920 A1
(43) Pub. Date: Nov. 9, 2023

(54) Systems and Methods for Increasing Hypnotizability

(71) Applicant: The Board of Trustees of the Leland Stanford Junior University, Stanford, CA (US)

(72) Inventors: Nolan R. Williams, Half Moon Bay, CA (US); David Spiegel, Palo Alto, CA (US); John Coetzee, San Francisco, CA (US)

(73) Assignee: The Board of Trustees of the Leland Stanford Junior University, Stanford, CA (US)

(21) Appl. No.: 18/314,005

(22) Filed: May 8, 2023

Related U.S. Application Data
(60) Provisional application No. 63/364,329, filed on May 6, 2022.

(51) Int. Cl. A61M 21/02; A61N 2/00; A61N 2/02

(52) U.S. Cl. CPC A61M 21/02; A61N 2/006; A61N 2/02

(57) ABSTRACT

Systems and methods for increasing hypnotizability in accordance with embodiments of the invention are illustrated. One embodiment includes a method of treating a recipient using hypnotherapy, including applying neurostimulation to a recipient's brain such that functional connectivity between an anterior cingulate cortex (ACC) and a dorsolateral prefrontal cortex (DLPFC) of the recipient's brain is increased, and activity in the ACC is decreased, and applying hypnotherapy in order to treat a condition of the recipient. In a further embodiment, the condition is selected from the group consisting of depression, anxiety, phobia, pain, stress, disordered eating, sleep disorders, posttraumatic stress, and addiction.

### CROSS-REFERENCE TO RELATED APPLICATIONS

The current application claims the benefit of and priority under 35 U.S.C. § 119(e) to U.S. Provisional Patent Application No. 63/364,329 entitled “Systems and Methods for Increasing Hypnotizability” filed May 6, 2022. The disclosure of U.S. Provisional Patent Application No. 63/364,329 is hereby incorporated by reference in its entirety for all purposes.

### STATEMENT OF FEDERALLY SPONSORED RESEARCH

This invention was made with Government support under contract AT009305 awarded by the National Institutes of Health. The Government has certain rights in the invention.

### FIELD OF THE INVENTION

The present invention generally relates to making humans more susceptible to being hypnotized, namely increasing hypnotizability via stimulation of specific brain structures.

### BACKGROUND

Hypnosis is a condition involving focused attention, reduced peripheral awareness, and an enhanced capacity to respond to suggestion. Hypnosis has been used medically throughout history. Hypnotherapy is the use of hypnosis in psychotherapy and is used by medical professionals to treat many different types of problems such as, but not limited to: depression, anxiety, phobias, pain, stress, eating disorders, sleep disorders, posttraumatic stress, and various addictions, e.g. smoking, food, gambling, alcoholism, etc.

Transcranial magnetic stimulation (TMS) is a type of brain stimulation that can be applied non-invasively. TMS is often applied using magnetic coils that create a focused magnetic field. The magnetic field in turn can be used to stimulate neurons via electromagnetic induction.

### SUMMARY OF THE INVENTION

Systems and methods for increasing hypnotizability in accordance with embodiments of the invention are illustrated. One embodiment includes a method of treating a recipient using hypnotherapy, including applying neurostimulation to a recipient's brain such that functional connectivity between an anterior cingulate cortex (ACC) and a dorsolateral prefrontal cortex (DLPFC) of the recipient's brain is increased, and activity in the ACC is decreased, and applying hypnotherapy in order to treat a condition of the recipient.

In a further embodiment, the condition is selected from the group consisting of depression, anxiety, phobia, pain, stress, disordered eating, sleep disorders, posttraumatic stress, and addiction.

In still another embodiment, the neurostimulation is repetitive paired associative stimulation with the DLPFC and the ACC.

In a still further embodiment, the neurostimulation is sequential continuous theta burst stimulation (cTBS) to the DLPFC followed by sequential cTBS to the ACC.

In yet another embodiment, the neurostimulation is repetitive paired associative stimulation with transcranial magnetic stimulation to the DLPFC and Vagus nerve stimulation to the ACC.

In a yet further embodiment, the neurostimulation is quadrapulse transcranial magnetic stimulation to the DLPFC.

In another additional embodiment, the neurostimulation is quadrapulse transcranial magnetic stimulation to the ACC.

In a further additional embodiment, the neurostimulation includes inhibitory low intensity focused ultrasound to the ACC.

In another embodiment again, the neurostimulation further includes inhibitory low intensity focused ultrasound to the DLPFC.

In a further embodiment again, the inhibitory low intensity ultrasound includes between 600 and 1800 ultrasound bursts delivered in 10-millisecond bursts repeated every 200 milliseconds using a fundamental frequency between 300 and 700 kHz, a pulse repetition frequency of 5 Hz, and a duty cycle of between 1% and 5%.

In yet another embodiment includes a hypnotherapy system, including a brain scanner configured to obtain a scan of a recipient's brain, a neuronavigator configured to generate at least one stimulation target based on the scan, and a brain stimulation device configured to deliver a neurostimulation to the at least one stimulation target, where the stimulation pattern increases hypnotizability by increasing functional connectivity between an anterior cingulate cortex (ACC) and a dorsolateral prefrontal cortex (DLPFC) of the recipient's brain, and decreasing activity in the ACC, where the recipient is subsequently provided hypnotherapy in order to treat a condition.

In still yet another embodiment, the condition is selected from the group consisting of depression, anxiety, phobia, pain, stress, disordered eating, sleep disorders, posttraumatic stress, and addiction.

In a still yet further embodiment, the neurostimulation is repetitive paired associative stimulation with the DLPFC and the ACC.

In still another additional embodiment, the neurostimulation is sequential continuous theta burst stimulation (cTBS) to the DLPFC followed by sequential cTBS to the ACC.

In a still further additional embodiment, the neurostimulation is repetitive paired associative stimulation with transcranial magnetic stimulation to the DLPFC and Vagus nerve stimulation to the ACC.

In still another embodiment again, the neurostimulation is quadrapulse transcranial magnetic stimulation to the DLPFC.

In a still further embodiment again, the neurostimulation is quadrapulse transcranial magnetic stimulation to the ACC.

In yet another additional embodiment, the neurostimulation includes inhibitory low intensity focused ultrasound to the ACC.

In a yet further additional embodiment, the neurostimulation further includes inhibitory low intensity focused ultrasound to the DLPFC.

In yet another embodiment again, the inhibitory low intensity ultrasound includes between 600 and 1800 ultrasound bursts delivered in 10-millisecond bursts repeated every 200 milliseconds using a fundamental frequency between 300 and 700 kHz, a pulse repetition frequency of 5 Hz, and a duty cycle of between 1% and 5%.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 illustrates a hypnotherapy system in accordance with an embodiment of the invention.

FIG. 2 illustrates a hypnotherapy controller in accordance with an embodiment of the invention.

FIG. 3 is a flow chart illustrating a hypnotherapy process in accordance with an embodiment of the invention.

### DETAILED DESCRIPTION

Turning now to the drawings, systems and methods for increasing hypnotizability are described. Hypnotherapy, where a medical professional induces a hypnosis state in a recipient in order to treat them, is a useful tool for health professionals. However, not every recipient can achieve a hypnotic state easily or at all, even if they would like to be treated. Such a recipient can be referred to as “not hypnotizable” or having “low hypnotizability.”

Conventionally, if a recipient has low hypnotizability, alternative treatment options are explored. Systems and methods described herein provide an additional option: increasing the hypnotizability of the recipient via specific stimulation of the brain. In general, increasing hypnotizability can be achieved by increasing functional connectivity between the anterior cingulate cortex (ACC) and the dorsolateral prefrontal cortex (DLPFC), while also inhibiting activity in the ACC. Increasing functional connectivity while also inhibiting activity in the same region can be difficult. As can be readily appreciated, each person's brain is idiosyncratic. What works for one brain might not work as well for a different brain. Therefore, multiple different stimulation patterns are described herein which can achieve the desired neurological changes. Once hypnotizability in a recipient is increased, hypnotherapy can take place and be used to treat any number of different conditions. As such, systems and methods described herein can be referred to as “hypnotherapy systems” and “hypnotherapy methods.”

### Hypnotherapy Systems

Hypnotherapy systems can increase the hypnotizability of a human. In many embodiments, hypnotherapy systems target specific areas of the brain with specific stimulation in order to achieve an increase in hypnotizability. In numerous embodiments, neuronavigation systems can be integrated with hypnotherapy systems in order to generate personalized stimulation targets for a particular recipient. In various embodiments, hypnotherapy systems include brain scanning machines such as (but not limited to) magnetic resonance imaging (MRI) machines, functional MRI (fMRI) machines, computed tomography (CT) scans, positron emission tomography (PET) scans, and/or any other brain scanning device as appropriate to the requirements of specific applications of embodiments of the invention. Brain stimulation devices such as (but not limited to) transcranial magnetic stimulation (TMS) machines and/or transcranial electric stimulation (TES) machines can be used to deliver stimulation in accordance with the parameters discussed herein. However, in various embodiments, other noninvasive and invasive stimulation methodologies can be used to provide stimulation.

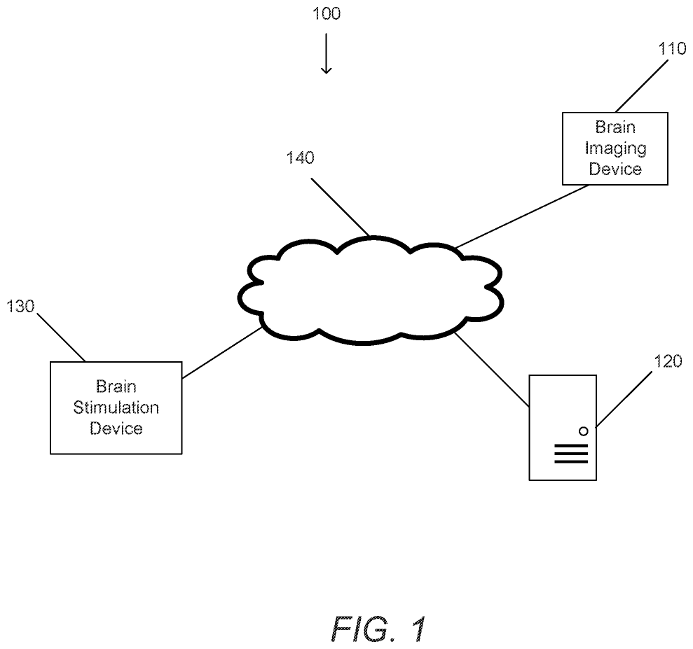

Turning now to FIG. 1, a hypnotherapy system in accordance with an embodiment of the invention is illustrated. Hypnotherapy system 100 includes a brain imaging device 110, a neuronavigation system 120, and a brain stimulation device 130. Devices in system 100 can communicate via a network 140. The network can be any network or set of networks such as (but not limited to), the Internet, an intranet, a wide area network, a local area network, and/or any other network infrastructure as appropriate to the requirements of specific applications of embodiments of the invention. However, in many embodiments, communication takes place via physical movement of data between devices using storage media.

In various embodiments, additional devices can be added to hypnotherapy systems. For example, hypnotherapy devices can be added for recipients to treat themselves (or be treated using) structured hypnosis. While a particular system architecture is illustrated in FIG. 1, any number of different architectures can be used as appropriate to the requirements of specific applications of embodiments of the invention. For example, in a variety of embodiments, neuronavigation systems and brain stimulation devices can be integrated into a single unit. However, any number of different architectures are possible without departing from the scope or spirit of the invention.

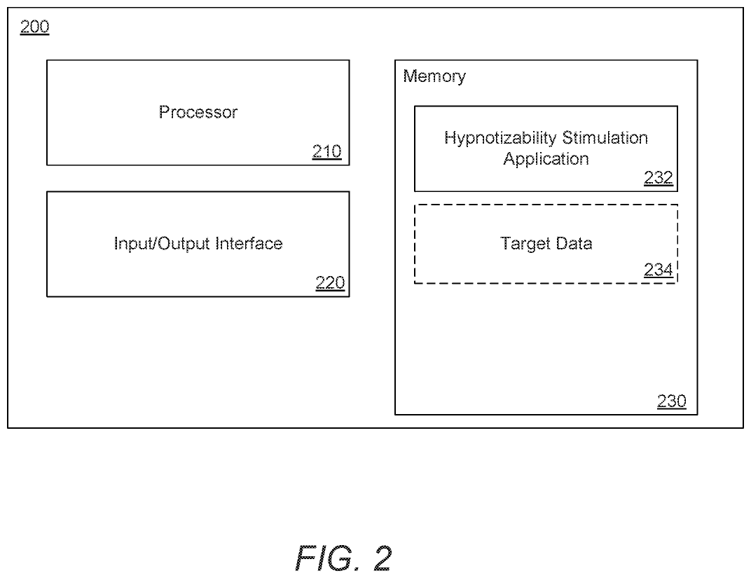

In many embodiments, the brain stimulation device includes a controller which is used to control the application of the stimulation. Turning now to FIG. 2, a controller for a brain stimulation device in accordance with an embodiment of the invention is illustrated. The controller 200 includes a processor 210. The processor can be any number of logic processing units including, but not limited to central processing units (CPUs), graphics processing units (GPUs), application-specific integrated circuits (ASICs), field-programmable gate arrays (FPGAs), and/or any logic processing circuitry and/or combination thereof. The controller 200 further includes an input/output (I/O) interface (220). I/O interfaces can be used to transmit data between connected devices, either wirelessly or via wired connections.

The controller 200 further includes a memory 230. Memory 230 can be implemented using volatile and/or nonvolatile storage media. The memory 230 contains a hypnotizability stimulation application 232 which can direct the processor to control the application of stimulation using the brain stimulation device. In many embodiments, the memory 230 also contains target data 234 which describes a specific target in a specific recipient's brain for stimulation to be applied to. In many embodiments, the target is obtained from a neuronavigation system. As can be readily appreciated, controllers can be implemented in any number of ways, and can be separate pieces of hardware from the brain stimulation device as well. Hypnotizability stimulation processes for increasing hypnotizability are discussed further below.

### Hypnotizability Stimulation and Hypnotherapy Processes

Hypnotizability stimulation processes can be used to increase the hypnotizability of a specific recipient. In many embodiments, hypnotizability stimulation processes include applying specific stimulation patterns to a recipient which increase functional connectivity between the anterior cingulate cortex (ACC) and the dorsolateral prefrontal cortex (DLPFC), while also inhibiting activity in the ACC. In various embodiments, the target area is a specific subregion of the ACC and/or DLPFC depending on the recipient.

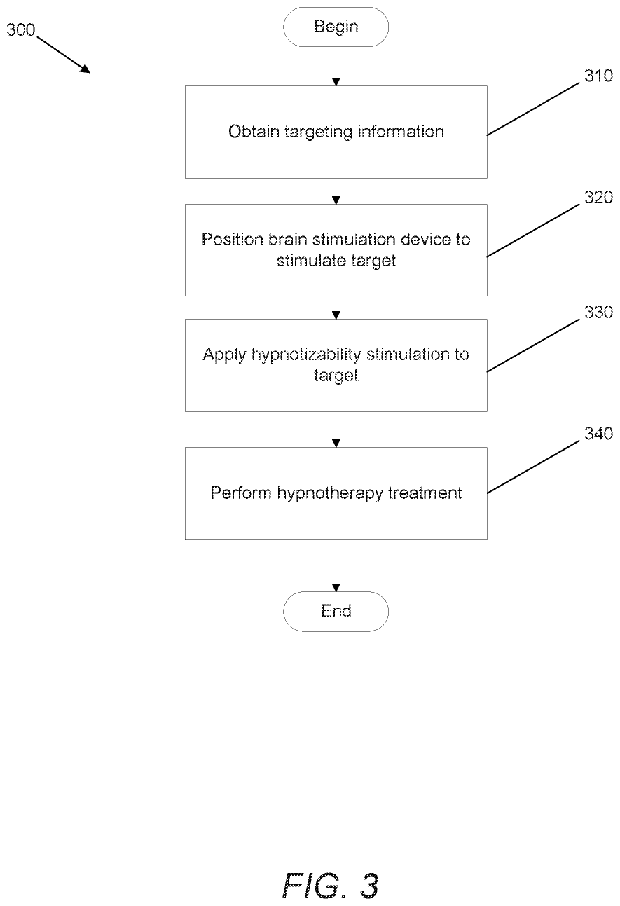

Turning now to FIG. 3, a flow chart illustrating a hypnotizability stimulation process in accordance with an embodiment of the invention is illustrated. Process 300 includes obtaining (310) targeting information. In many embodiments, the targeting information describes a specific location (or locations) in the recipient's brain to stimulate. Targeting information can further include data describing stimulation patterns from a set of stimulation patterns which are most likely to produce the desired results based on the recipient's specific neural circuitry.

A brain stimulation device is positioned (320) to stimulate the target. In many embodiments, this involves positioning a TMS coil at the proper position over the recipient's head. However, the positioning can differ based on the type of brain stimulation device used. Hypnotizability stimulation is then applied (330) to the target. In many embodiments, stimulation is applied over a period of time which may involve multiple sessions of stimulation, some of which may occur on different days. Stimulation patterns and protocols are discussed in further detail below. After hypnotizability has been increased, the recipient can be treated (340) using hypnotherapy.

In many embodiments, treatment can be applied using an automated, interactive hypnotherapy application. Hypnotherapy applications such as (but not limited to) the Reveri app by Reveri Health, Inc. of Stinson Beach, California, and/or any other interactive hypnotherapy program can be used as appropriate to the requirements of specific applications of embodiments of the invention. In numerous embodiments, the interactive hypnotherapy program can be tailed to the problems being addressed. In various embodiments, hypnotizability stimulation can be provided using a wearable and/or implantable stimulation device in communication, which can be in communication with the interactive hypnotherapy program. For example, a recipient may use an at-home brain stimulation device in conjunction with an interactive hypnotherapy application in order to have an enhanced response.

As can be readily appreciated, the specific steps of applying stimulation to a recipient's brain may significantly differ based on the brain stimulation modality. In some embodiments, targeting data may not necessarily be personalized, and generalized brain anatomy can be used to position the brain stimulation device. Further, depending on the recipient, only an increase in functional connectivity or an inhibition of activity may be achieved. Hypnotizability simulation is discussed below.

### Hypnotizability Stimulation

As noted above, hypnotizability stimulation attempts to increase hypnotizability by increasing functional connectivity between the ACC and the DLPFC, while also inhibiting activity in the ACC. In order to achieve this, a number of different stimulation patterns can be used, including (but not limited to): repetitive paired associative stimulation with DLPFC-ACC; sequential continuous theta burst stimulation (cTBS) to DLPFC then ACC; sequential TBS to ACC then DLPFC; repetitive paired associative stimulation with TMS to the DLPFC and Vagus nerve stimulation (VNS) to ACC; quadrapulse to the DLPFC; and quadrapulse to ACC.

Repetitive paired associated stimulation with the DLPFC-ACC can be delivered using a number of different stimulation methodologies including (but not limited to) TMS. In many embodiments, active rapid-rate paired associative stimulation (rPAS) including a repetitive pairing of TMS of the ACC/dorsolateral prefrontal cortex (DLPFC) with TMS of the DLPFC with an interstimulus interval of between 1 and 25 ms can be used. TMS pulses can be delivered at a stimulus strength sufficient to induce a peak-to-peak 1 mV motor-evoked potential.

Sequential cTBS to the DLPFC then the ACC, and sequential cTBS to the dACC then the DLPFC can be delivered, where the cTBS involves delivering pulse trains of 20 Hz to 70 Hz pulses, at 3 Hz to 7 Hz, for anywhere between 500 and 1000 pulses. In many embodiments, the pulse train includes 3 pulses, although other amounts of pulses per train are usable. In various embodiments, a session of cTBS includes 3-pulse trains of 30 Hz pulses at 6 Hz for 44 seconds for a total of 800 total pulses.

Repetitive paired associated stimulation with TMS to the DLPFS and VNS to the ACC can be achieved using active rPAS consisting of a repetitive pairing of electrical stimulation of the vagus nerve at the left neck (10-360 pulses at 0.1 Hz) with TMS of the contralateral DLPFC with an interstimulus interval of 1-25 ms.

Quadrapulse stimulation (QPS, sometimes quadripulse or quadropulse) can be delivered to the DLPFC and/or the ACC. QPS includes four-pulse trains where each pulse is delivered at the same intensity and are separated by 1.5 ms, repeatedly given at 0.2 Hz.

In numerous embodiments, low intensity focused ultrasound (LIFU) can be used as a stimulation method. When applied in accordance with the below parameters, LIFU can achieve either an excitatory or an inhibitory effect on brain tissue. While for certain applications, a single session might be enough for an increase in immediate hypnotizability (useful for soon-after hypnosis sessions), in various embodiments more stimulation is delivered over a longer period of time to have a more lasting change in brain dynamics. In various embodiments, the ultrasound is delivered in 10-30 minute sessions, either unilaterally or bilaterally. In some embodiments, 1-10 sessions are performed per day, with a 40-50 minute interval between sonication of the same brain structure, for between 1 and 5 days per week, for up to 2 weeks. In numerous embodiments, more than one day of stimulation is provided. Excitatory sonication parameters are:

Excitatory LIFU

| | | |
|---|---|---|
| Fundamental Frequency | 300-700 | kHz |
| Amplifier Intensity | 10-30 | W |
| Pulse Repetition Frequency | 5 | Hz |

| | |
|---|---|
| Duty Cycle | 5%-15% |
| Total Duration (start to finish) | 10 minutes-30 minutes |
| Pattern | 20-milliseond ultrasound bursts repeated |
| | every 200 miliseconds. Burst for 2 |
| | seconds, followed by an 8 second interval |
| Total Bursts | 600-1800 |

Inhibitory LIFU

| | | |
|---|---|---|
| Fundamental Frequency | 300-700 | kHz |
| Amplifier Intensity | 10-30 | W |
| Pulse Repetition Frequency | 5 | Hz |

| | |
|---|---|
| Duty Cycle | 1%-5% |
| Total Duration (start to finish) | 2 minutes-12 minutes |
| Pattern | 10-millisecond ultrasound bursts repeated |
| | every 200 milliseconds |
| Total Bursts | 600-3600 |

In many embodiments, the ultrasound does not exceed 720 kW/cm³ at the target in accordance with current FDA safety guidelines. However, higher outputs can be functionally equivalent so long as they do not induce heating greater than 2 degrees Celsius in the brain tissue. In numerous embodiments, due to the penetrating power of ultrasound, just the ACC may be inhibited without interacting directly with the DLPFC. In various embodiments, this enables the patient to have minimal impact to executive control.

Furthermore, other brain structures may be implicated in hypnotizability. Stimulation parameters described above can be applied to said structures to increase hypnotizability as appropriate to the requirements of specific applications of embodiments of the invention for a given patient, including (but not limited to), stimulation of the linsula and posterior cingulate cortex (PCC).

Although specific methods of increasing hypnotizability are discussed above, many different methods can be implemented in accordance with many different embodiments of the invention. For example, while a number of parameters are discussed above with respect to different stimulation methods, different parameters within tolerable ranges for particular recipients may be utilized as appropriate without departing from the scope or spirit of the invention. Further, other parameters may be used for similar effect that are appropriate for alternative stimulation methods such as transcranial direct current stimulation (tDCS), or other neurostimulation modalities. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method of treating a recipient using hypnotherapy, comprising:
applying neurostimulation to a recipient's brain such that functional connectivity between a anterior cingulate cortex (ACC) and a dorsolateral prefrontal cortex (DLPFC) of the recipient's brain is increased, and activity in the ACC is decreased;
and applying hypnotherapy in order to treat a condition of the recipient.

2. The method of claim 1, wherein the condition is selected from the group consisting of: depression, anxiety, phobia, pain, stress, disordered eating, sleep disorders, posttraumatic stress, and addiction.

3. The method of claim 1, wherein the neurostimulation is repetitive paired associative stimulation with the DLPFC and the ACC.

4. The method of claim 1, wherein the neurostimulation is sequential continuous theta burst stimulation (cTBS) to the DLPFC followed by sequential cTBS to the ACC.

5. The method of claim 1, wherein the neurostimulation is repetitive paired associative stimulation with transcranial magnetic stimulation to the DLPFC and Vagus nerve stimulation to the ACC.

6. The method of claim 1, wherein the neurostimulation is quadrapulse transcranial magnetic stimulation to the DLPFC.

7. The method of claim 1, wherein the neurostimulation is quadrapulse transcranial magnetic stimulation to the ACC.

8. The method of claim 1, wherein the neurostimulation comprises inhibitory low intensity focused ultrasound to the ACC.

9. The method of claim 8, wherein the neurostimulation further comprises inhibitory low intensity focused ultrasound to the DLPFC.

10. The method of claim 8, wherein the inhibitory low intensity ultrasound comprises between 600 and 1800 ultrasound bursts delivered in 10-millisecond bursts repeated every 200 milliseconds using: a fundamental frequency between 300 and 700 kHz; a pulse repetition frequency of 5 Hz, and a duty cycle of between 1% and 5%.

11. A hypnotherapy system, comprising:
a brain scanner configured to obtain a scan of a recipient's brain;
a neuronavigator configured to generate at least one stimulation target based on the scan; and
a brain stimulation device configured to deliver a neurostimulation to the at least one stimulation target, where the stimulation pattern increases hypnotizability by increasing functional connectivity between an anterior cingulate cortex (ACC) and a dorsolateral prefrontal cortex (DLPFC) of the recipient's brain, and decreasing activity in the ACC;
where the recipient is subsequently provided hypnotherapy in order to treat a condition.

12. The system of claim 11, wherein the condition is selected from the group consisting of: depression, anxiety, phobia, pain, stress, disordered eating, sleep disorders, posttraumatic stress, and addiction.

13. The system of claim 11, wherein the neurostimulation is repetitive paired associative stimulation with the DLPFC and the ACC.

14. The system of claim 11, wherein the neurostimulation is sequential continuous theta burst stimulation (cTBS) to the DLPFC followed by sequential cTBS to the ACC.

15. The system of claim 11, wherein the neurostimulation is repetitive paired associative stimulation with transcranial magnetic stimulation to the DLPFC and Vagus nerve stimulation to the ACC.

16. The system of claim 11, wherein the neurostimulation is quadrapulse transcranial magnetic stimulation to the DLPFC.

17. The system of claim 11, wherein the neurostimulation is quadrapulse transcranial magnetic stimulation to the ACC.

18. The system of claim 11, wherein the neurostimulation comprises inhibitory low intensity focused ultrasound to the ACC.

19. The system of claim 18, wherein the neurostimulation further comprises inhibitory low intensity focused ultrasound to the DLPFC.

20. The system of claim 18, wherein the inhibitory low intensity ultrasound comprises between 600 and 1800 ultrasound bursts delivered in 10-millisecond bursts repeated every 200 milliseconds using: a fundamental frequency between 300 and 700 kHz; a pulse repetition frequency of 5 Hz, and a duty cycle of between 1% and 5%.
